(19) United States
(12) Patent Application Publication
(10) Pub. No.: US 2008/0243139 A1
(43) Pub. Date: Oct. 2, 2008

(54) Method for assembling a ring used in a small pupil phaco procedure

(76) Inventor: Vaclav Dusek, Bellevue, WA (US)

Correspondence Address: IRELL & MANELLA LLP, 840 NEWPORT CENTER DRIVE, SUITE 400, NEWPORT BEACH, CA, 92660, US

(21) Appl. No.: 12/074,731

(22) Filed: Mar. 5, 2008

Related U.S. Application Data
(60) Provisional application No. 60/918,404, filed on Mar. 15, 2007.

(51) Int. Cl. A61F 9/007

(52) U.S. Cl. 606/107; 600/236

(57) ABSTRACT

A ring that can maintain a pupil in an extended position during an ophthalmic procedure. The ring has a plurality of loops that capture iris tissue. The ring is configured to extend the pupil when iris tissue is inserted into each loop. An ophthalmic procedure such as phacoemulsification can then be performed on the patient. The ring has a first end and a second end that each have an indent. The ring includes an adhesive that extends into the indents and attaches the first end to the second end.

### CROSS-REFERENCE TO RELATED APPLICATION

This application claims priority to Application No. 60/918,404 filed on Mar. 15, 2007.

### BACKGROUND OF THE INVENTION

1. Field of the Invention

The present invention relates to a ring used in a ophthalmic surgical procedure.

2. Background Information

There are various ophthalmic procedures that require the dilation of the pupil. For example, cataracteous lenses are typically replaced in a procedure commonly referred to as phacoemulsification or phaco for short. In a phaco procedure the lens is broken up with an instrument, typically with an ultrasonically driven tool. The instrument has an aspiration port that aspirates the broken lens material from the patient's ocular chamber.

It is desirable to extend the pupil during a phaco procedure to provide the surgeon with a wide view of the lens. One technique for extending the pupil includes pulling back the iris with a series of plastic hooks. It is has been found that using plastic hooks can cause damage to iris tissue.

### BRIEF SUMMARY OF THE INVENTION

A ring used to maintain a pupil in an extended position during an ophthalmic procedure. The ring has a first end, a second end and a plurality of loops. The first end has a first indent and the second end has a second indent. The ring also includes an adhesive that extends into the first and second indents and attaches the first end to the second end.

### BRIEF DESCRIPTION OF THE DRAWINGS

FIG. 1 is an illustration of a ring of the present invention;

FIG. 2 is an illustration showing an enlarged view of the ring;

FIG. 3 is an illustration showing iris tissue being inserted into a first loop of the ring;

FIG. 4 is an illustration showing iris tissue being inserted into a second loop of the ring;

FIG. 5 is an illustration showing the iris tissue within gaps of the loops;

FIG. 6 is an illustration showing a pupil being maintained in an extended position by the ring;

FIG. 7 is a perspective view of an injector and ring plate used to load and inject the ring.

### DETAILED DESCRIPTION

Described is a ring that can maintain a pupil in an extended position during an ophthalmic procedure. The ring has a plurality of loops that capture iris tissue. The ring is configured to extend the pupil when iris tissue is inserted into each loop. An ophthalmic procedure such as phacoemulsification can then be performed on the patient. The ring has a first end and a second end that each have an indent. The ring includes an adhesive that extends into the indents and attaches the first end to the second end.

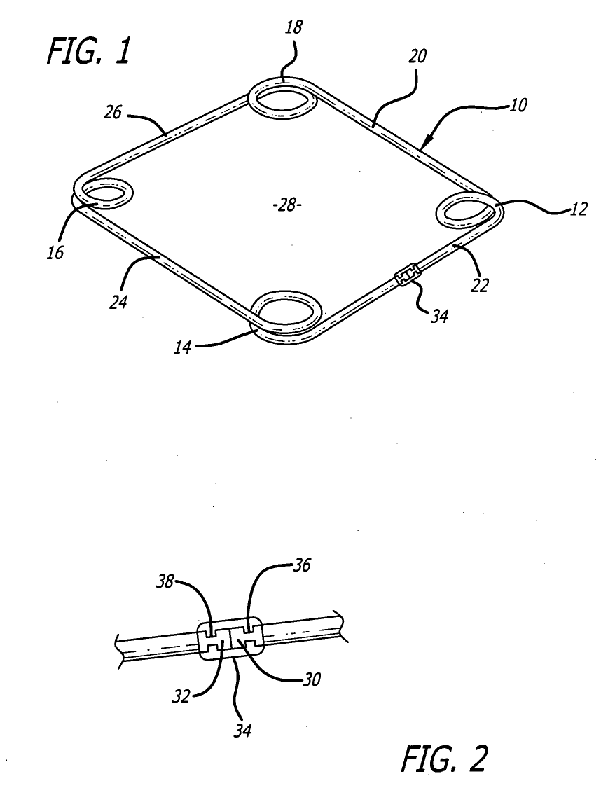

Referring to the drawings more particularly by reference numbers, FIG. 1 shows an embodiment of a ring 10 that can be used to extend a pupil during an ophthalmic procedure. The ring 10 has a plurality of loops 12, 14, 16 and 18 located at the corners of four sides 20, 22, 24 and 26. Each loop 12, 14, 16 and 18 may be formed by one full turn. Although one full turn is shown and described, it is to be understood that each loop 12, 14, 16 and 18 may have multiple turns. The four sides 20, 22, 24 and 26 circumscribe a center opening 28.

The ring 10 preferably has a square configuration such that the sides 20, 22, 24 and 26 are of equal dimension. Although a square ring is shown and described, it is to be understood that the ring may have a rectangular configuration where all sides 20, 22, 24 and 26 are not of equal dimension. Additionally, the ring may have a non-rectangular shape. For example, the ring 10 may be shaped as a triangle that has three sides and three loops located at the ring corners. Although three and four sided rings have been described, it is to be understood that the ring may have any number of sides and loops. The ring 10 is preferably constructed from a molded plastic material, although it is to be understood that other materials such as metal, or plastic coated metal may be employed.

FIG. 2 shows a preferred embodiment for constructing the ring 10. One side 20 of the ring 10 has two ends 30 and 32 that are butt attached by an adhesive 34. Each end 30 and 32 may have an indent 36 and 38, respectively. The adhesive 34 can flow into the indents 36 and 38 to increase the strength of the butt attachment of the ring 10. The indents 36 and 38 create surface structure that minimizes shearing and de-lamination of the adhesive 34 from the ring 10. By way of example, the adhesive 34 may be a biocompatible material such as Class VI epoxy. The adhesive 34 can be applied with a tool (not shown) that insures a repeatable volume and dimensions of the solidified adhesive form.

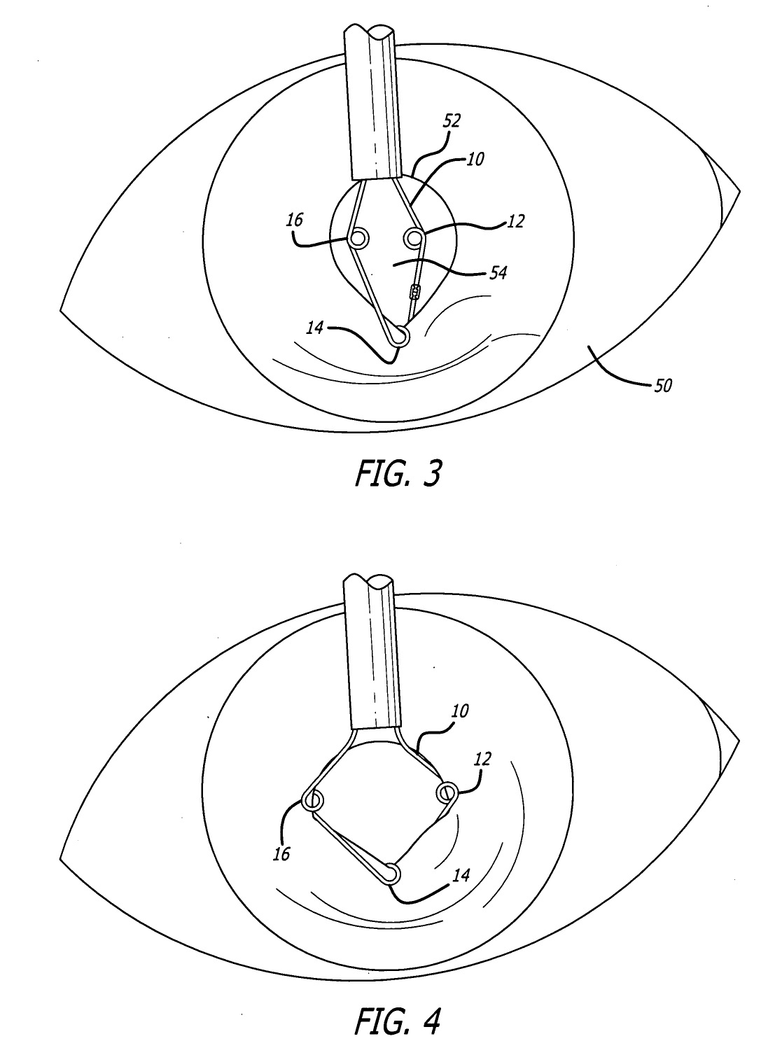

FIG. 3 shows the initial stages of the ring 10 being inserted into a patient's eye 50 to stretch the iris 52 and extend the pupil 54. A tool 56 such as a forcep can be used to pull the iris so that iris tissue is inserted into the first loop 12 of the ring 10. As shown in FIG. 4, the ring 10 can be manipulated so that iris tissue is inserted into loops 12 and 16.

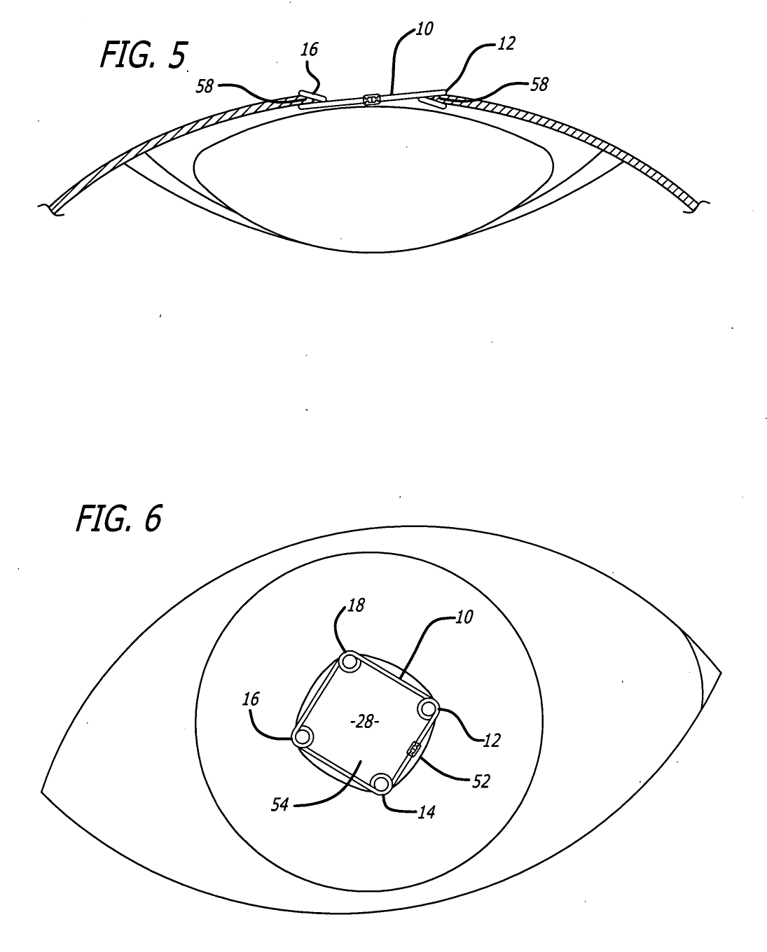

As shown in FIG. 5 each loop 12 and 14 has a gap 58 that receives and captures iris tissue. The loop design provides an easy means of inserting and capturing iris tissue. The flexibility of the ring 10 allows the loops to deflect and apply a clamping force onto the iris tissue. The clamping force assist in maintaining the position of the ring relative to the eye.

As shown in FIG. 6 iris tissue can be inserted into the second 14 and fourth 18 loops to fully stretch the iris 52 and extend the pupil 54. An ophthalmic procedure can then be performed on the eye. For example, a phaco procedure can be performed wherein the lens is emulsified and aspirated from the eye. The ring 10 maintains the pupil 54 in the fully extended position while the center opening 28 provides a wide viewing area during the procedure. When the procedure is complete one of the sides 20, 22, 24 or 26 can be cut with an instrument and the ring 10 can be removed from the eye.

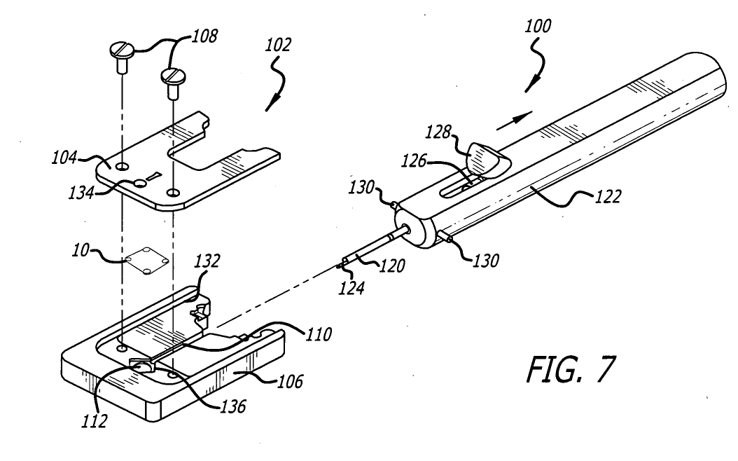

FIG. 7 shows an embodiment of an injector 100 that can be used to inject a ring 10 into a patient's eye. The ring 10 can be loaded into the injector 100 with the use of a ring holder 102. The ring holder 102 may include a cover 104 that is attached to a base plate 106 by fasteners 108. The base plate 106 has a channel 110 and a recess 112. The recess 112 receives the ring 10.

The injector 100 includes a cannula 120 attached to a handle 122. Within the cannula 120 is a wire hook 124. The wire hook 124 is connected to an inner slide tube 126 located within the handle 122. A button 128 is attached to the inner slide tube 126. The injector 100 may also have a pair of guide pins 130 that are attached to the handle 122 and cooperate with corresponding channel features 132 of the base plate 106 to properly align the injector 100 when the cannula 120 is inserted into the base plate channel 110.

In operation, the cannula 120 is inserted into the base plate channel 110. When fully inserted the wire hook 124 extends to approximately the center of the ring 10. The cover 104 may have an opening 134 that allows an operator to visually see the hook 124 within the ring opening. An operator then pulls the button 128 in the direction indicated by the arrow. Pulling the button 128 causes the hook 124 to grasp the ring loops and pull the ring 10 into the cannula 120. The recess 112 has tapered walls 136 to assist in the ring collapsing within the channel 112 for insertion into the cannula 120. Once loaded, the ring 10 can be injected into a patient's eye by pushing the button 128 in the opposite direction.

While certain exemplary embodiments have been described and shown in the accompanying drawings, it is to be understood that such embodiments are merely illustrative of and not restrictive on the broad invention, and that this invention not be limited to the specific constructions and arrangements shown and described, since various other modifications may occur to those ordinarily skilled in the art.

What is claimed is:

1. A ring used to maintain a pupil in an extended position during an ophthalmic procedure, comprising:
a ring that has a plurality of loops, said ring has a first end and a second end, said first end has a first indent and said second end has a second indent; and,
an adhesive that extends into said first and second indents and attaches said first end to said second end of said ring.

2. The ring of claim 1, wherein said ring is shaped as a rectangle.

3. The ring of claim 1, wherein said ring is shaped as a square.

4. The ring of claim 1, wherein each loop has at least one full turn.

5. The ring of claim 2, wherein said ring has four loops.

6. The ring of claim 1, wherein said adhesive is constructed from a biocompatible material.

7. A method for assembling a ring that is used to maintain a pupil an extended position for an ophthalmic procedure, comprising:
providing a ring that has a plurality of loops, a first end and a second end, the first end has a first indent and the second end has a second indent; and,
applying an adhesive that that extends into the first and second indents and attaches the first end to the second end of the ring.

8. The method of claim 7, wherein the adhesive is constructed from a biocompatible material.

9. An injector for injecting an ophthalmic ring, comprising:
a handle;
a cannula attached to said handle;
a slide tube within said handle;
a hook attached to said slide tube; and,
a button attached to said slide tube.

10. The injector of claim 9, further comprising a ring holder with a recess and a channel that can receive said cannula.

11. A method for loading an ophthalmic ring into an injector, comprising:
inserting a cannula of a injector into a channel of a ring holder, the ring holder containing a ring; and,
actuating the injector to pull the ring into the cannula.

12. The method of claim 11, wherein actuation causes a hook to pull the ring into the cannula.

13. The method of claim 11, wherein actuation is caused by moving a button.
